(19) United States
(12) Patent Application Publication
(10) Pub. No.: US 2006/0173324 A1
(43) Pub. Date: Aug. 3, 2006

(54) 3d imaging system and method for signaling an object of interest in a volume of data

(75) Inventors: Claude Cohen-Bacrie, New York, NY (US); Jean-Michel Lagrange, Moissy Cramayel (FR); Nicolas Villain, Clamart (FR); Claire Levrier, Rueil-Malmaison (FR); Robert Randall Entrekin, Paris (FR)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., Eindhoven (NL)

(21) Appl. No.: 10/545,591

(22) PCT Filed: Mar. 1, 2004
(86) PCT No.: PCT/IB04/00634

§ 371 Date: Aug. 15, 2005

(30) Foreign Application Priority Data
Mar. 13, 2003 (FR) 0303120

(51) Int. Cl. A61B 8/14

(52) U.S. Cl. 600/440

(57) ABSTRACT

The invention concerns a medical imaging system comprising means (2) of acquiring at least one volume of 3D data (3DV), means (3) of detecting at least one object of interest in said volume of data, display means (4) able to supply a 2D representation (2DR) of said volume of data and signaling means (5) intended to signal a location of said object of interest by means of a signal (SIG) superimposed on said 2D representation.

### FIELD OF THE INVENTION

The present invention relates to a medical imaging system intended to form a 2D representation of an object of interest from an acquisition of a volume of 3D data. It also relates to a method implemented by such a system. Finally, it relates to a computer program product implementing such a method.

It finds an application especially in the medical field, in particular for ultrasonic imaging and magnetic resonance imaging.

### BACKGROUND OF THE INVENTION

3D imaging systems have developed a great deal during the past few years, including in the medical field. Consequently a doctor is more and more induced to make a diagnosis, for example to seek an object of interest, from a volume of 3D data, a 2D representation of which he views on a screen. Such a volume comprises more information than a simple 2D image and makes it possible to detect objects of interest that can scarcely be discerned on a 2D image. On the other hand, it is also more difficult to manipulate. This is because, unlike an image, not all the data are simultaneously available on a single 2D representation of the volume. The doctor is to navigate in the volume and display several different 2D representations of this volume. He therefore needs an increased amount of time to scan the volume exhaustively and make his diagnosis.

### SUMMARY OF THE INVENTION

It is an aim of the present invention to propose a solution for making the visual detection by a user of an object of interest within a volume of 3D data more reliable and more rapid, in particular in the medical field.

This aim is achieved by a medical imaging system comprising:
acquisition means intended to acquire at least one volume of 3D data,
means of detecting at least one object of interest in said volume of data, intended to supply characteristics of said object,
means of displaying said volume of data intended to provide a 2D representation of said volume,
signaling means intended to signal a location of said object of interest from said characteristics, using a signal superimposed on said 2D representation.

The system according to the invention signals to the user, by means of a sound or a color, that he is displaying a 2D representation comprising a possible location of the object of interest. Such signals attract his attention to this possible location of the object of interest. The user can possibly move accordingly in the volume of 3D data in order to display the object of interest at another angle. This signaling is particularly advantageous in the case where, as in the medical field, the object of interest is often difficult to detect to the naked eye and may not be detected by a doctor. Alerted to all the locations where one or more objects of interest may be situated, the doctor can concentrate his energy on the observation of a 2D representation rather than on navigation in the 3D volume. The system according to the invention therefore has the advantage of guiding the user when he is navigating in the volume of 3D data. Such a system also has the advantage of sparing the user from having to navigate in the volume exhaustively.

Another advantage of the system according to the invention is to inject into a 2D representation of a volume of 3D data characteristics related to the object of interest which cannot be obtained from the 2D representation alone but require on the contrary apprehending the volume of 3D data as a whole. This is the case, for example, when the region of interest is liable to comprise objects which are both spherical and tubular in shape. A 2D representation of the 3D volume may in this case exhibit a more or less circular cross-section of the object, making it difficult to distinguish between a spherical object and a tubular object. The detection means according to the invention are able to supply a characteristic of the object of interest such as its orientation. Such a characteristic enables the user to recognize a tubular object having a favored orientation from a spherical object not having any particular orientation.

### BRIEF DESCRIPTION OF THE DRAWINGS

The invention will be further described with reference to examples of embodiments shown in the drawings to which, however, the invention is not restricted.

FIG. 1 presents a functional diagram of an ultrasonic imaging system according to the invention,

FIG. 2 illustrates the effect of a subtractive median filter used by the detection means of the system according to the invention, in the case of a 1D profile,

FIG. 3 illustrates the principle used by the derivation sub-means according to the invention, in the case of a non-noisy 1D profile,

FIG. 4 illustrates the principle used by the derivation sub-means according to the invention, in the case of a noisy profile,

FIG. 5a presents an example of a tubular object of interest and the orientation of the particular vectors of the structure tensor supplying the principal axes of the object,

FIG. 5b presents a possible choice of a display axis and of three orthogonal views for constructing a 2D representation of a volume of 3D data according to the invention,

FIG. 6 presents an example of a 2D representation of a volume of 3D data according to the invention,

FIG. 7 presents an example of microcalcification signaled in a 2D representation of a volume of 3D data according to the invention,

FIG. 8 presents an example of a tubular structure signaled in a 2D representation of a volume of 3D data according to the invention,

FIG. 9 presents a functional diagram of a magnetic resonance imaging system according to the invention,

FIG. 10 presents three contrast change curves in a delimited zone of a region of interest over time.

### DESCRIPTION OF PREFERRED EMBODIMENTS

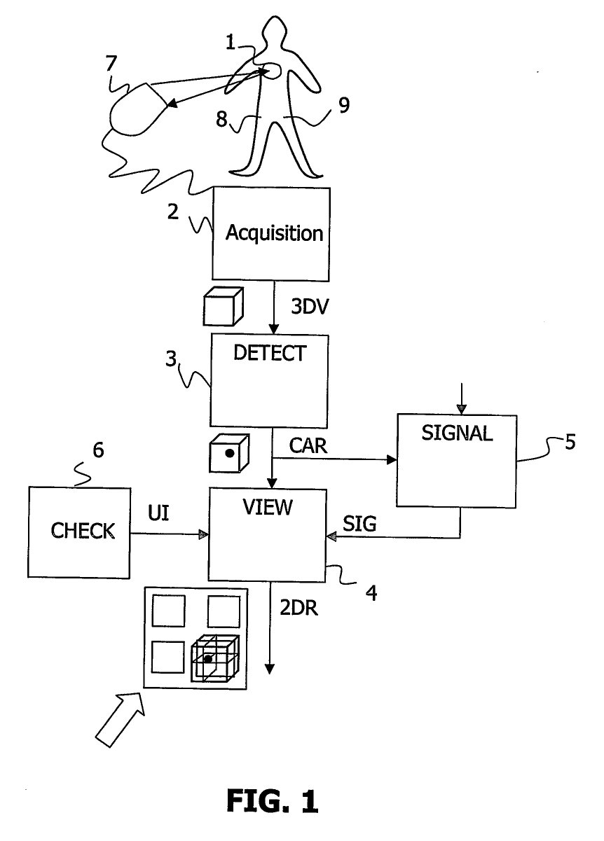

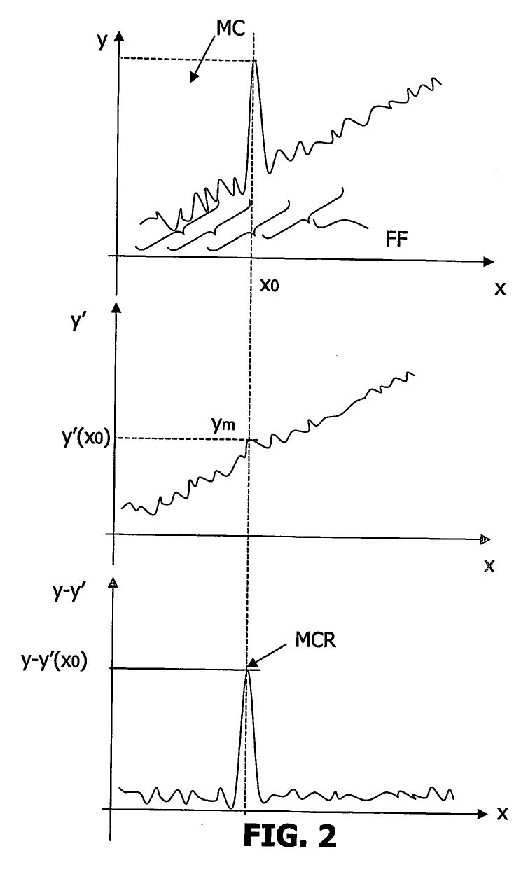

FIG. 1 depicts a functional diagram of a 3D imaging system according to the invention, in the medical field. In a first embodiment, an ultrasonic imaging system for the detection of microcalcifications of the breast is considered. Such a system comprises means 2 of acquiring a volume 3DV of ultrasonic data 3D of a region of interest 1 of the human body, for example a breast, means 3 of detecting objects of interest, for example microcalcifications MC, in said volume 3DV, display means 4 intended to deliver a 2D representation 2DR of the volume 3DV and means 5 of signaling the microcalcifications MC in the representation 2DR.

The acquisition means 2 are able to emit ultrasonic signals 8 in the direction of the region of interest 1 by means of a probe 7 and to receive delayed ultrasonic signals 9 in return, the said delayed signals being returned by the region of interest 1. The probe 7 comprises elements which are capable of converting an electrical pulse into a sound wave and to receive a response returned by the region of interest. The said elements can be assembled in a matrix in order to form a two-dimensional probe or in an array to form a one-dimensional probe. If the probe is a matrix of elements, a 3D volume of ultrasonic data is acquired directly. In the case of a 1D probe, that is to say an array of elements, conventional echographic imaging, according to a method known to persons skilled in the art, provides, for a given position of the probe, an image representing a 2D section of the environment in the plane of the probe. By then moving the probe, several sections through the same environment are obtained. All these sections constitute a 3D volume of data.

The volume 3DV obtained supplies a cartography of the ultrasonic energy returned by the environment formed by the region of interest. The region of interest is liable to comprise zones which return more or less energy. It is said that these zones are more or less echogenic. Some objects of interest, such as microcalcifications MC, are point-source objects, very echogenic, which appear as small bright points in the volume 3DV. One difficulty in locating these microcalcifications in the volume 3DV is that they are generally masked by a noise called “speckle”, which makes them difficult to detect with the naked eye.

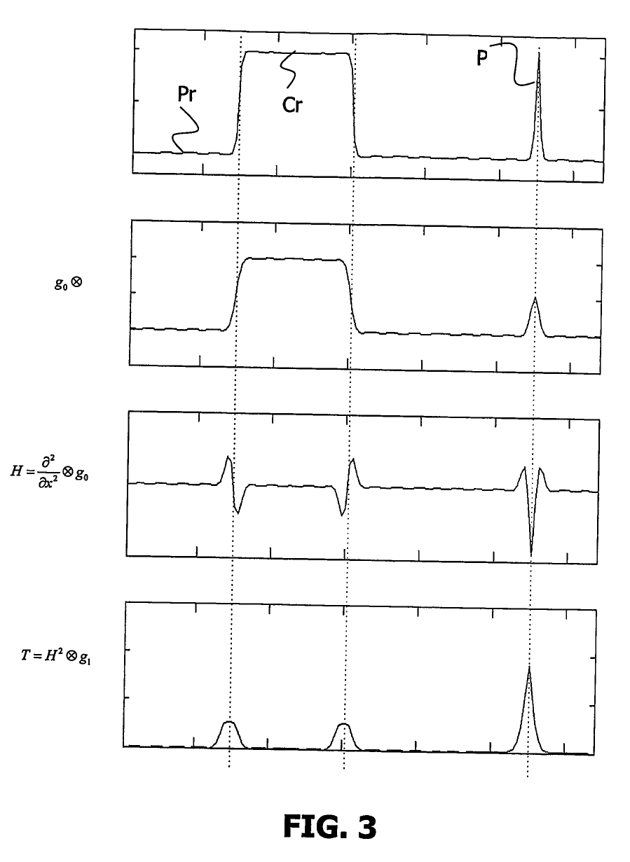

The system according to the invention comprises detection means 3 intended to detect objects of interest in the volume 3DV of ultrasonic data. In one embodiment of the invention, the said detection means 3 comprise median filtering sub-means, which consist of applying a subtractive median filter to the volume of data 3DV in order to enhance objects of interest of small size such as microcalcifications. FIG. 3 illustrates the principle of subtractive median filtering in the 1D case. A profile y(x) of a microcalcification MC is depicted therein. The microcalcification MC forms a narrow peak surrounded by peaks of lesser intensity due to noise.

Effecting a median filtering at a point y(x₀) of the profile on a filtering window FF of width I consists of:
sorting the I values of the profile (y(x₀−½), . . . , y(x₀), . . . , y(x₀+½−1)),
extracting the median value yₘ,
taking the median value yₘ for the filtered value y′(x₀),
renewing the operation at each point on the profile.

The effect of such a filtering is to make the peak due to the microcalcification MC disappear, provided that the filtering window FF is sufficiently wide compared with the width of the peak. In a second step, the median profile y′ is subtracted from the original profile y, which has the effect of dispensing with the low-frequency variations in the profile whilst preserving the contrast at the microcalcification. The profile y-y′ reveals an enhanced microcalcification MCR.

In the case of a volume of data such as the volume 3DV, a median filter 3D is used. In this case, the filtering window FF is a rectangular parallelepiped, for example a cube. Its size is chosen according to a template of objects of interest sought. Because of the non-ideal response of the imaging system, a point-source object is represented by a spot which is not necessarily isotropic, that is to say which may be deformed in some directions rather than in others. To take account of this defect in focusing, it may be necessary to consider a non-cubic parallelepipedal filtering window.

A volume of filtered data is obtained in which the structures corresponding to the template are enhanced. The detection means according to the invention comprise thresholding sub-means intended to extract the structures with the highest contrast from amongst the enhanced structures. The threshold is in particular chosen according to the power of the noise present in the ultrasonic data. After thresholding, a location of the structures retained is easily derived. As a characteristic CAR of an object of interest detected, the detection means according to the invention supply for example a position (xₒᵢ, yₒᵢ, zₒᵢ) of the object of interest in a reference frame (O,x,y,z) of the volume 3DR.

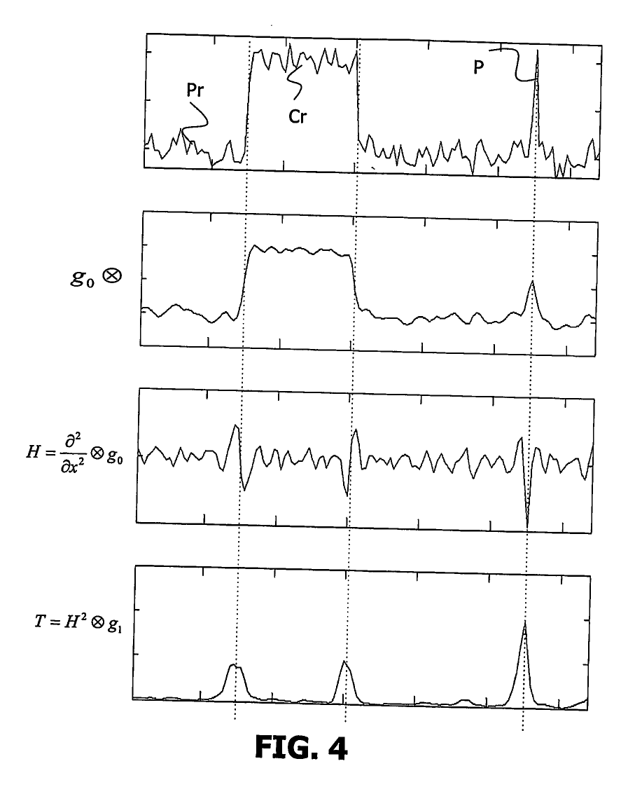

In a second embodiment of the invention, objects of interest of elongate shape, for example tubular, are sought. In the field of medical imaging, it is a case for example of blood vessels, milk ducts, ligaments etc. In the case of echography of the breast, it is advantageous to be able to locate the objects of interest of elongate shape in order to exclude them from potential microcalcifications and to know their orientation. In order to detect such anisotropic structures, the detection means 3 according to the invention comprise sub-means of deriving the volume of data 3DV. The principles used by the said derivation sub-means are illustrated by FIG. 3 in the case of a non-noisy 1D profile Pr and by FIG. 4 in the case where the profile Pr is noisy. A Gaussian convolution kernel g₀ is first of all applied to the profile Pr so as to filter the noise. According to a technique known to persons skilled in the art, said derivation means then consist of calculating a second derivative, in order to reveal an object of interest having a contrast peak in the profile Pr. This is because, since a first derivative is canceled out at the location of the crests sought, a second derivative is preferred, since it has a maximum absolute value at the location of the said crests. This second derivative is then squared and then post-filtered by a Gaussian kernel g₁. It is used to detect the presence of a crest, that is to say a one-dimensional contrast peak. An example of a peak P and a square wave Cr is presented in FIGS. 3 and 4. It is clear that the second derivative enhances the peak P and to a lesser extent detects the edges of the square wave Cr whilst considerably reducing the power of the noise.

In three dimensions the detection means 3 make it necessary to calculate all the second derivatives along the three axes x,y,z of the reference frame (O, x, y, z), which makes it possible to derive the Hessian matrix associated with all the points of the volume 3DV:

$$H = [ \begin{matrix}\frac{{δ}^{2}}{δ {x}^{2}} ⊗ {g}_{0} \frac{{δ}^{2}}{δ x δ y} ⊗ {g}_{0} \frac{{δ}^{2}}{δ x δ z} ⊗ {g}_{0} \\ \frac{{δ}^{2}}{δ y δ x} ⊗ {g}_{0} \frac{{δ}^{2}}{δ {y}^{2}} ⊗ {g}_{0} \frac{{δ}^{2}}{δ y δ z} ⊗ {g}_{0} \\ \frac{{δ}^{2}}{δ z δ x} ⊗ {g}_{0} \frac{{δ}^{2}}{δ z δ y} ⊗ {g}_{0} \frac{{δ}^{2}}{δ {z}^{2}} ⊗ {g}_{0}\end{matrix} ]$$

A tensor of structure T=(H.H^{T})\textcircled{×}g₁ is next calculated. A thresholding of the trace of the tensor T makes it possible to retain the structures with the highest contrast corresponding amongst other things to the tubular structures sought. The threshold is chosen according to a statistic of the noise liable to interfere with the trace of the tensor.

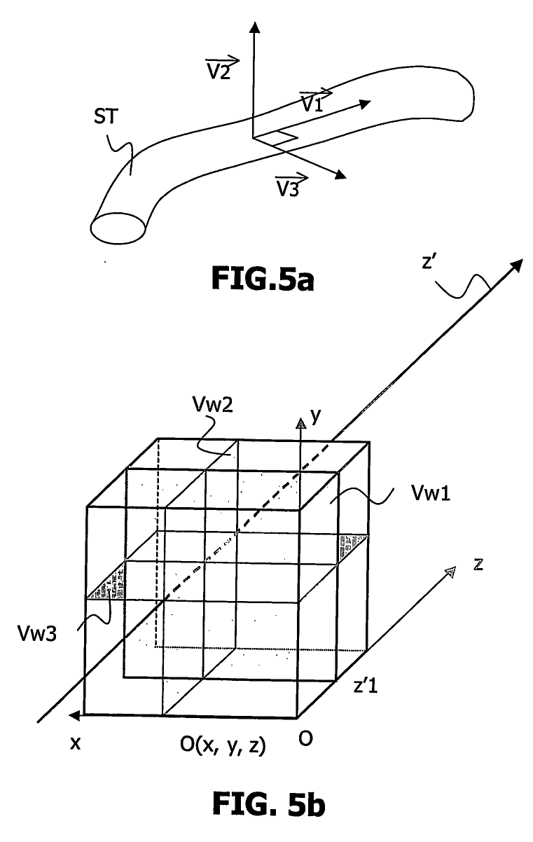

The tensor T being a positive defined matrix, it has three real positive proper values λ₁, λ₂ and λ₃, with λ₁<λ₂<λ₃, associated with three proper vectors [symbol] ₁, [symbol] ₂ and [symbol] ₃ forming a proper base aligned on the object of interest. An example of a tubular structure is presented in FIG. 5a. The proper vector [symbol] ₁ associated with the smallest proper value λ₁ indicates the direction of the object of interest in the case of a tubular object. The said derivation sub-means also make it possible to assess whether the object of interest is isotropic or anisotropic from ratios between proper values:
if λ₁≈λ₂≈₃ and λ₁ is large, the object of interest is highly contrasted and has no favored direction. This is known as a blob,
if λ₃/λ₁ is large, the object of interest has a favored direction,
if λ₁≈λ₂ and λ₃ is large, the object of interest is a plane.

The object of interest can then be characterized not only by a location (xₒᵢ, yₒᵢ, zₒᵢ) but also by an orientation. This orientation is for example given by the proper vector [symbol] ₁. It is also possible to calculate a measurement of angle α between [symbol] ₁ and a vector normal to a section through the volume 3DV.

The display means 4 of the imaging system according to the invention form a 2D representation 2DR of the volume of 3D data. In a preferred embodiment of the invention, the 2D representation 2DR comprises 3 orthogonal sections or views Vw₁, Vw₂ and Vw₃. These three views are defined along a display axis z′ in the following manner:
the view Vw₁ is orthogonal to the axis z′ and cuts the volume at a depth z₀′,
the views Vw₂ and Vw₃ are orthogonal to each other and to the view Vw₁ and pass through the axis z′.

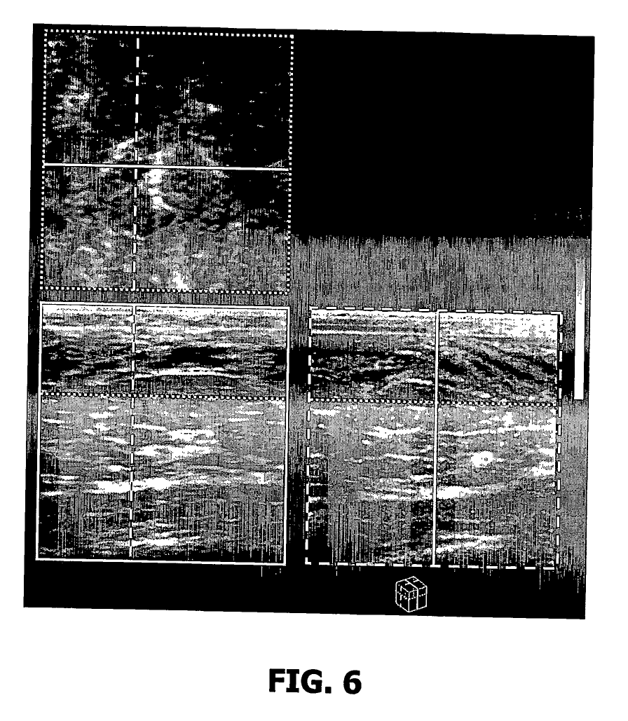

FIG. 5b illustrates a possible choice of the display axis z′ and of the three orthogonal views Vw₁, Vw₂ and Vw₃. An example of a 2D representation 2DR is presented in FIG. 6. It should be noted that the display axis z′ is not necessarily parallel to the axis z of the reference frame (O, x, y, z).

In the preferred embodiment, the imaging system according to the invention comprises check means 6 for checking a position of the said display axis in the said volume 3DV and a position of said first view Vw₁ along said axis z′. The positions of the other two views Vw₂ and Vw₃ are modified accordingly. The user can therefore navigate in the volume by choosing a position of the display axis z′ and a position of the view Vw₁ on this axis. When he quickly varies the coordinate z′ of the view Vw₁ he obtains a sequence called a “cineloop”.

The signaling means 5 of the imaging system according to the invention are intended to signal a location of said object of interest in said 2D representation, by means of a signal SIG superimposed on the representation 2DR. It is a case of alerting a user to the presence of an object of interest in the volume 3DV and more precisely indicating to him that the object of interest is visible on the representation 2DR which it is in the process of displaying. To do this, the signaling means use the characteristics CAR supplied by the detection means 3.

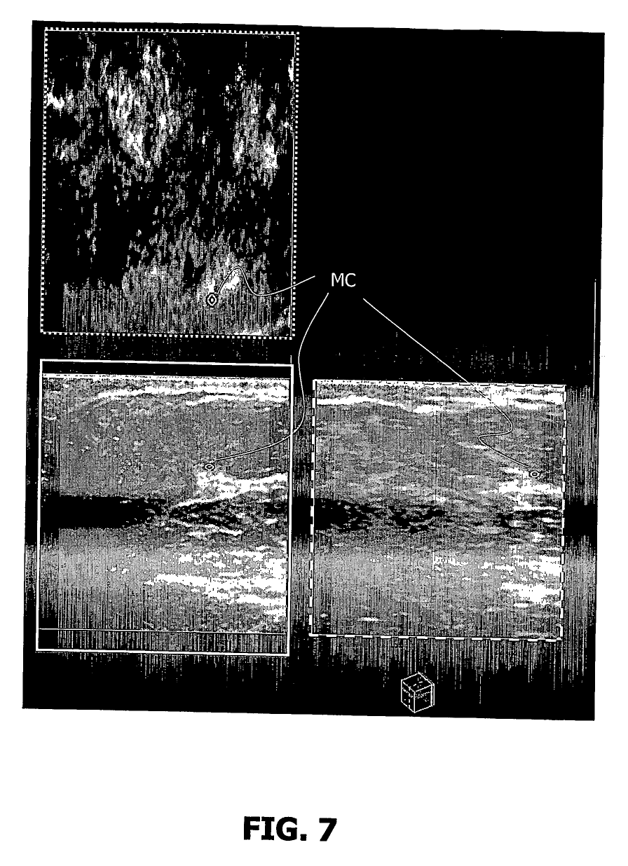

In the case of an isotropic object of interest, for example a microcalcification, the characteristics CAR supplied by said detection means may be a location defined by coordinates in the reference frame (O, x, y, z). The signaling means 5 then consist of superimposing the signal SIG on the representation 2DR when said location is included in one of the three views Vw₁, Vw₂ or Vw₃ contained in the representation 2DR. This signal SIG may be visual and appear on the view concerned as a colored shape, for example a circle centered on said location, as shown by FIG. 7 for a microcalcification MC. It may equally well be audible, that is to say a bleep is emitted when the user defines, using the check means, a representation 2DR where one of the views cuts the object of interest.

It should be noted that any other signal SIG able to alert the user may be used, for example a flash.

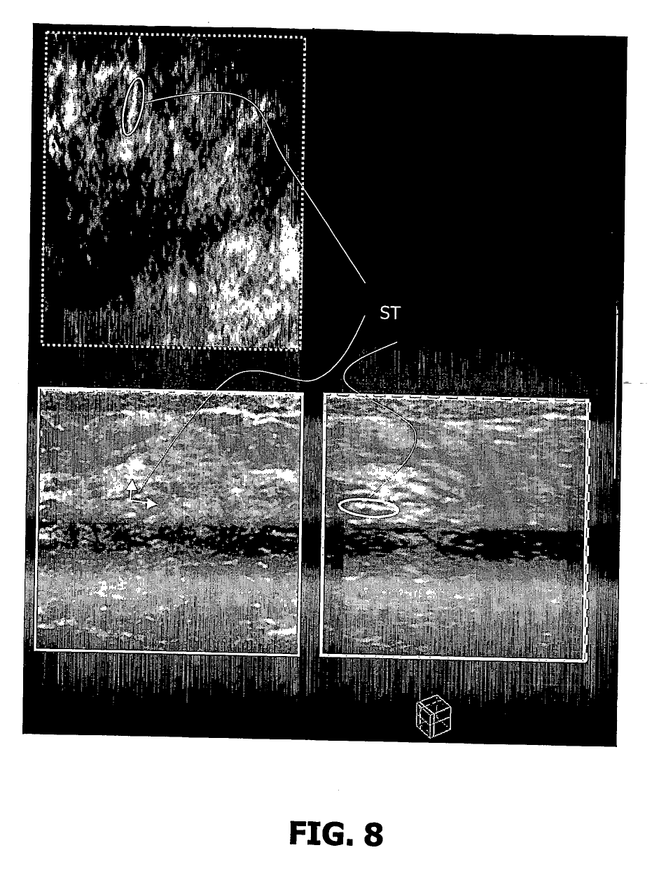

In the case of an anisotropic object having an orientation, for example a blood vessel, the signal SIG may be an arrow representing the orientation of said vector or a color coding the measurement of angle α superimposed on a section 2DR of the volume 3DR, as shown by FIG. 8 for a tubular structure ST.

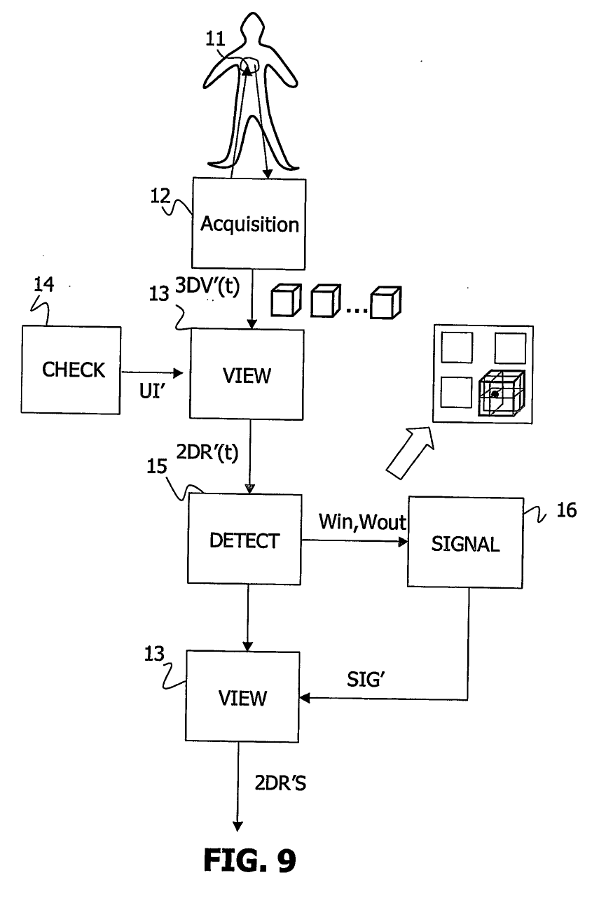

In a third embodiment of the invention, a magnetic resonance imaging system presented in FIG. 9 is considered. Magnetic resonance imaging uses a variable magnetic field. By a principle known to persons skilled in the art, the response of the environment studied to this excitation is recorded by the system and a sequence of sections of the region of interest is acquired, so as to form a volume of 3D data.

Such a system makes it possible to display soft tissues. It is in particular used for imaging the breast and detecting any mammary lesions. For this purpose, the acquisition means 12 are able to effect a dynamic acquisition of n volumes of data 3DV′(t), t=t₀, t₁ . . . tₙ₋₁ at n discrete times.

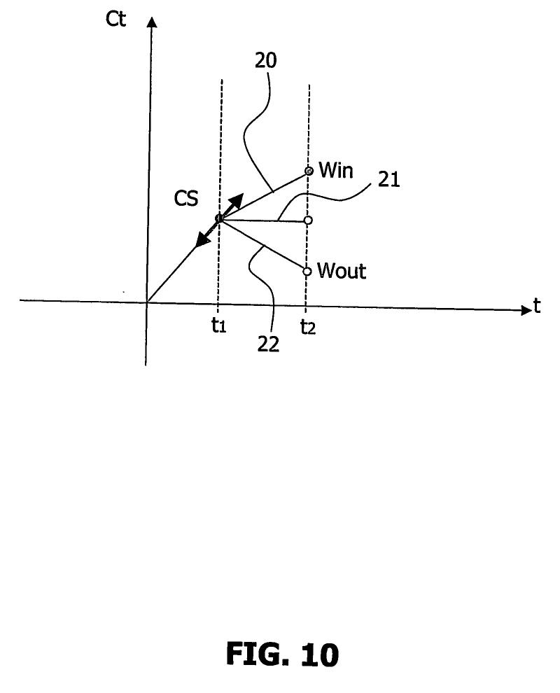

The dynamic acquisition aims to follow the diffusion of a contrast product, generally gadolinium, within the region of interest. This product, injected at time t₀, has the property of creating a contrast flash in a highly perfused zone of the region of interest, for example a mammary lesion. It is said that the lesion “adopts the contrast”. However a lesion adopts the contrast differently depending on whether it is a case of a benign or malignant lesion. In other words, the speed at which the contrast product invades and leaves the lesion is not the same whatever the type of lesion encountered. It is therefore advantageous to look at the propagation of the contrast product at successive times t₀, t₁, t₂ . . . tₙ₋₁ and to assess its dynamics over time. Between times to and ti, adoption of contrast is referred to. The contrast product progressively invades the region of interest and emphasizes any lesions. FIG. 10 depicts examples of curves of change in contrast Ct in the region of interest. Between times t₁, and t₂, three main scenarios are possible:
either the quantity of contrast product present at the point of the lesion continues to increase, as indicated by the curve 20. A phenomenon of wash-in (Wᵢₙ) is referred to and, in this case, a benign lesion is often involved,
or the quantity of contrast product stagnates, as indicated by the curve 21,
or the quantity of contrast product falls, as indicated by the curve 22. A phenomenon of wash-out (Wₒᵤₜ) is referred to and, in this case, probably a malignant lesion is involved.

The display means 13 of the system according to the invention enable the doctor to display one or more volumes of data 3DV(t) obtained at different times t in the form of sections through this volume. The check means 14 enable him to choose a section Vw₁′(t) where he has isolated an object of interest, for example a lesion, and thus to display the change in contrast on this section.

The purpose of the detection means 15 is to reveal any phenomena of wash-in and wash-out. Said means comprise local mean calculation means. It may be a case either of a spatial mean on the chosen 2D sections Vw₁′(t) at all points on the said sections, or a spatial mean on the volumes 3DV(t) at any point on said volumes. Said means also comprise sub-means of calculating the contrast slope between two successive times tᵢ and tᵢ₊₁, at all points on said sections or said volumes.

The sub-means of calculating the mean consist of evaluating a local mean. Consider a point on a volume 3DV′(t), the local mean at this point is obtained by summing, in a vicinity V centered on the point processed, all the values of points of the volume belonging to V sufficiently close to the value of the point. This requires extracting in the vicinity V a related sub-vicinity SV whose values meet homogeneity criteria and averaging the values contained in SV. There exist a great variety of approaches for extracting homogeneous zones in a vicinity. Such approaches involve segmentation techniques known to persons skilled in the art. The sub-means for calculating the slope effect the subtraction, between two consecutive times, of the values of local means described above and supply a measurement of the contrast slope, that is to say an evaluation of the speed of propagation of the contrast product in the area of interest between times t₁ and t₂. A positive slope between the times t₁ and t₂ indicates a wash-in phenomenon whilst a negative slope indicates a wash-out phenomenon.

The doctor generally displays a sequence Vw₁′(t) or a particular view of the sequence and the curve representing the contrast slope in parallel. The signaling means 16 of the system according to the invention make it possible to display directly an adoption or loss of contrast at any point on the section observed either by superimposing, or by displaying separately, a coloring whose code corresponds to the speed of propagation between two consecutive times. This makes it possible in particular to convert the wash-in and wash-out indices into signals which are superimposed on the section through the volume 3DV observed. For example, it is possible to color in red in the case of wash-in and blue in the case of wash-out. One advantage of the signaling means 16 according to the invention is to add time information to a 2D representation of an anatomical acquisition of a volume of data 3DV′(t₀). All the information available to it are calculated at every point in the volume and can therefore be grouped together on the same page for a given section through the volume 3DV, in order to facilitate the making of a diagnosis.

In a fourth embodiment, the system according to the invention comprises means of storing the volume of 3D data able to store said volume in the form of a collection of representations 2DR.

In the case of an ultrasonic imaging system as described in the first and second embodiments, the system according to the invention makes it possible in fact to define one or more representations 2DR revealing the object of interest. These representations 2DR have been defined by the doctor using the check means 6 and signaling means 5. It can be considered that these representations group together the data of the volume 3DV which are truly useful to the doctor in order to make a diagnosis and that said representations can advantageously be stored in place of the volume of 3DV or in addition to it.

In the case of a magnetic resonance imaging system as described in the third embodiment of the invention, it can be considered that the sequence Vw₁′(t) or even a particular image in this sequence, combined with the wash-in and wash-out indices, group together all the data useful to the doctor for making his diagnosis and can therefore advantageously supplement the n volumes of data 3DV′(t) or even replace them.

One advantage of the system according to the invention is therefore to afford savings in storage of the volumes of data 3DV or 3DV′(t) acquired.

The major advantage of said storage means is to facilitate any new access to the data. This is because, when a doctor wishes to consult a medical file comprising data obtained by means of a 3D imaging system, he is not obliged to waste time navigating in the volume 3DV. The representations 2DR which were stored concentrate all the useful data.

The invention is not limited to the embodiments which have just been described by way of example. Modifications or improvements can be made thereto whilst remaining within the scope of the invention. In particular, other imaging modes, such as X-ray imaging, can be used.

In the claims the verb “comprise” is used to signify that the use of other elements, means or steps is not excluded.

1. A medical imaging system comprising:
acquisition means intended to acquire at least one volume of 3D data,
means of detecting at least one object of interest in said volume of data, intended to supply characteristics of said object,
means of displaying said volume of data intended to provide a 2D representation of said volume including at least a portion of the detected object of interest,
signaling means intended to signal a location of said object of interest from said characteristics, using a signal superimposed on said 2D representation.

2. A system as claimed in claim 1, characterized in that said 2D representation comprises a first section through said volume of data, said first section being orthogonal to a display axis, a second section comprising said axis and orthogonal to the first section and a third section comprising said axis and orthogonal to the first and second sections, and in that said location of the object of interest is a zone of intersection of the object of interest with said first, second or third section.

3. A system as claimed in claim 2, characterized in that it comprises check means for checking a position of said display axis in said volume and a position of said first section along said axis.

4. A system as claimed in claim 2, characterized in that said signaling means are able to emit a sound in order to signal the presence of said object of interest in said 2D representation.

5. A system as claimed in claim 2, characterized in that said signaling means are able to mark said intersection zone by a color.

6. A system as claimed in claim 1, characterized in that said system is an ultrasonic imaging system.

7. A system as claimed in claim 6, characterized in that, the object of interest being a tubular object comprising an orientation, said detection means are able to supply a measurement of said orientation and said signaling means are able to signal the location of said object of interest on said 2D representation and its orientation.

8. A system as claimed in claim 1, characterized in that, said system being a magnetic resonance imaging system intended to follow a propagation of a contrast product in said region, said detection means are able to calculate a speed of propagation of said product through said object of interest and said signaling means are able to signal the location of said object on said 2D representation by superimposing on it a signal indicating said speed of propagation.

9. A system as claimed in claim 1, characterized in that it also comprises means of storing the volume of 3D data able to store said volume in the form of a collection of 2D representations, said 2D representations comprising said signal.

10. A method for displaying at least one object of interest in a medical diagnostic image, comprising the steps of:
acquiring at least one volume of 3D data,
detecting said object of interest in said volume of data in order to supply characteristics of said object,
displaying said volume of data in order to supply a 2D representation of said volume which includes at least a portion of said detected object of interest,
signaling for signaling a location of said object of interest in said 2D representation using said characteristics.

11. A computer program product for implementing a method as claimed in claim 10.

12. A system as claimed in claim 1, wherein the object of interest has a boundary within the volume of data; and
wherein the signaling means signals a location of said object of interest using a signal superimposed on said 2D representation within the boundary of the object of interest.

13. A system as claimed in claim 12, wherein the signal superimposed on said 2D representation produces a distinctive color within the boundary of the object of interest in comparison to surrounding anatomy.

14. A system as claimed in claim 12, wherein the signal superimposed on said 2D representation produces a distinctive contrast within the boundary of the object of interest in comparison to surrounding anatomy.
